(19) United States
(12) Patent Application Publication
(10) Pub. No.: US 2014/0088638 A1
(43) Pub. Date: Mar. 27, 2014

(54) Detachable End Effector with Lateral Lock

(71) Applicant: ETHICON ENDO-SURGERY, INC., Cincinnati, OH (US)

(72) Inventor: Shailendra K. Parihar, Mason, OH (US)

(73) Assignee: Ethicon Endo-Surgery, Inc., Cincinnati, OH (US)

(21) Appl. No.: 13/627,192

(22) Filed: Sep. 26, 2012

(51) Int. Cl. A61B 17/29; A61B 17/00

(52) U.S. Cl. 606/206; 606/1

(57) ABSTRACT

A surgical device comprises an elongate shaft comprising a distal end and a proximal end. An actuator is operably connected to the proximal end of the elongate shaft. The actuator may be a manual handle. An end effector is adapted for in vivo attachment to and detachment from the distal end of the elongate shaft. A loader is sized to receive the end effector. A lock extends laterally from the end effector and is operative to prevent the end effector from leaving the loader. The lock may move medially relative the end effector to allow the end effector to leave the loader.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While a variety of different minimally invasive surgical devices are known, no one has previously made or used the surgical devices and methods in accordance with the present invention.

### SUMMARY

In one embodiment, a surgical device comprises an elongate shaft comprising a distal end and a proximal end. An actuator is operably connected to the proximal end of the elongate shaft. The actuator may be a manual handle. An end effector is adapted for in vivo attachment to and detachment from the distal end of the elongate shaft. A loader is sized to receive the end effector. A lock extends laterally from the end effector and is operative to prevent the end effector from leaving the loader. The lock may move medially relative the end effector to allow the end effector to leave the loader.

In another embodiment, a surgical device comprises an elongate shaft having an outer tube and an inner rod positioned in the outer tube. The outer tube and inner rod each comprise a distal end and a proximal end. An actuator is operably connected to the proximal ends of the outer tube and the inner rod. An end effector is adapted for in vivo attachment to and detachment from the elongate shaft. The end effector comprises an outer casing comprising a first mating feature adapted to attach to the distal end of the outer tube; an inner shuttle axially moveable relative the outer casing, the inner shuttle comprising a second mating feature adapted to attach to the distal end of the inner rod; a pair of surgical jaws having an opened position and closed position dependant on the relative axial position of the shuttle and casing; and a prong moveable between a lateral position and a medial position. A loader comprises a shaft with a distal tube sized to receive the end effector. The distal tube comprises a recess adapted to receive the prong when it is in its lateral position thereby locking the end effector in the distal tube.

The surgical device may comprise a ring circumscribing the outer casing moveable between a proximal position and a distal position. The ring may actuate the prong between its lateral position to its medial position. A spring may bias the ring to its proximal position. The spring may also bias the shuttle distally. The ring may be actuated by the insertion of the inner rod into the surgical end effector. The prong may be attached to a leaf spring biasing the prong to its lateral position.

The first mating feature may comprise a medial tooth adapted to engage a notch in the outer tube. The second mating feature may comprise a medial tooth adapted to engage a notch in the inner rod. The second mating feature may comprise a pair of proximally oriented prongs each comprising a medially oriented tooth sized and adapted to engage a notch on the inner rod. The distal end of the outer tube may comprise a sloped surface adapted to laterally spread the proximally oriented prongs of the shuttle, thereby disengaging the medially oriented teeth from the notch on the inner rod.

In yet another embodiment, a surgical device comprises an elongate shaft having an outer tube and an inner rod positioned in the outer tube, the outer tube and inner rod each comprising a distal end and a proximal end. An actuator is operably connected to the proximal ends of the outer tube and the inner rod. A tubular loader is sized to receive the end effector. The tubular loader comprises a recess adapted to receive the prong when it is in its lateral position thereby locking the end effector in the tubular loader. An end effector is adapted for in vivo attachment to and detachment from the elongate shaft. The end effector comprises an outer casing and a means to attach the outer casing to the outer tube; an inner shuttle axially moveable relative the outer casing and a means to attach the shuttle to the inner rod; a pair of surgical jaws having an opened position and closed position dependant on the relative axial position of the shuttle and casing; and a means to lock and unlock the end effector in the tubular loader. The surgical device may further comprises a means to detach the inner rod from the shuttle.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts an example of a loader, end effector, shaft, and actuator;

FIG. 2 depicts an exploded view of the shaft;

FIG. 3 depicts a cross-sectional view of a portion of the shaft;

FIG. 4 depicts a perspective view of the end effector;

FIG. 5 depicts an exploded view of the end effector;

FIG. 6 depicts a cross-sectional view of the end effector;

FIG. 7 depicts a side view of the loader and end effector (not engaged with one another);

FIG. 8 depicts a cross-sectional view of the end effector locked in the loader; and

FIG. 9 depicts a cross-sectional view of the end effector attached to the shaft and unlocked in the loader.

### DETAILED DESCRIPTION

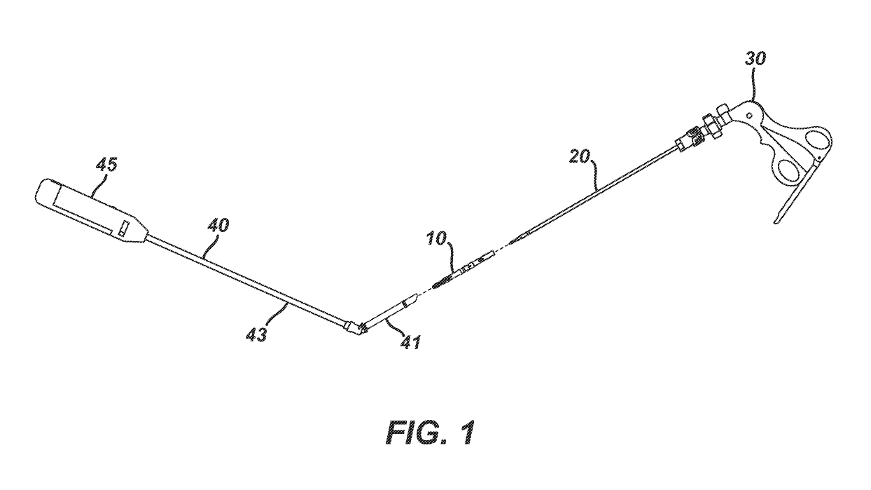

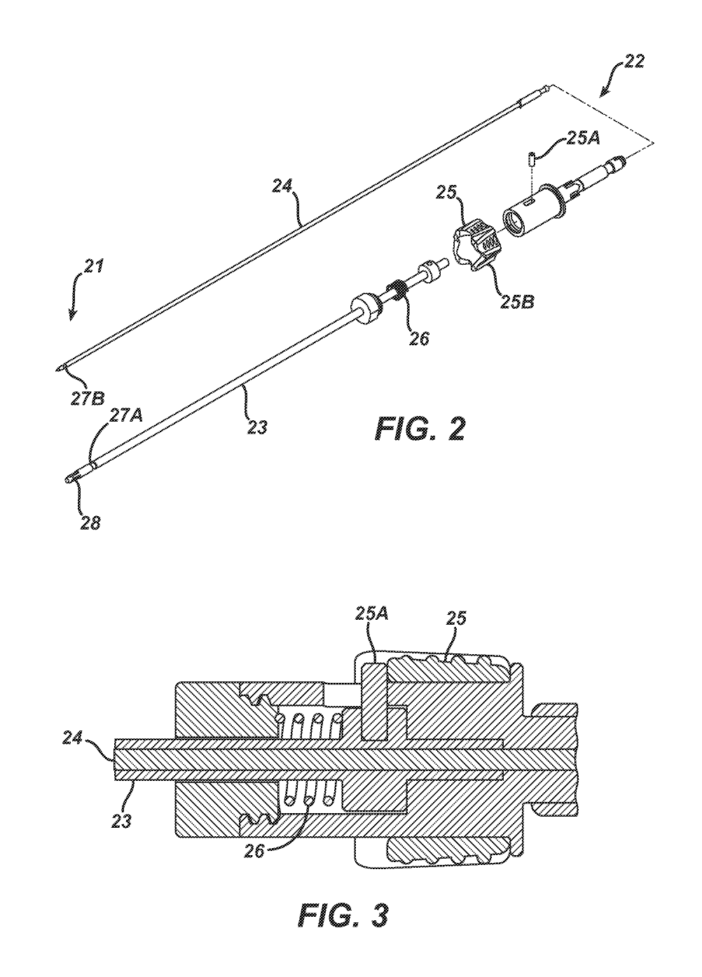

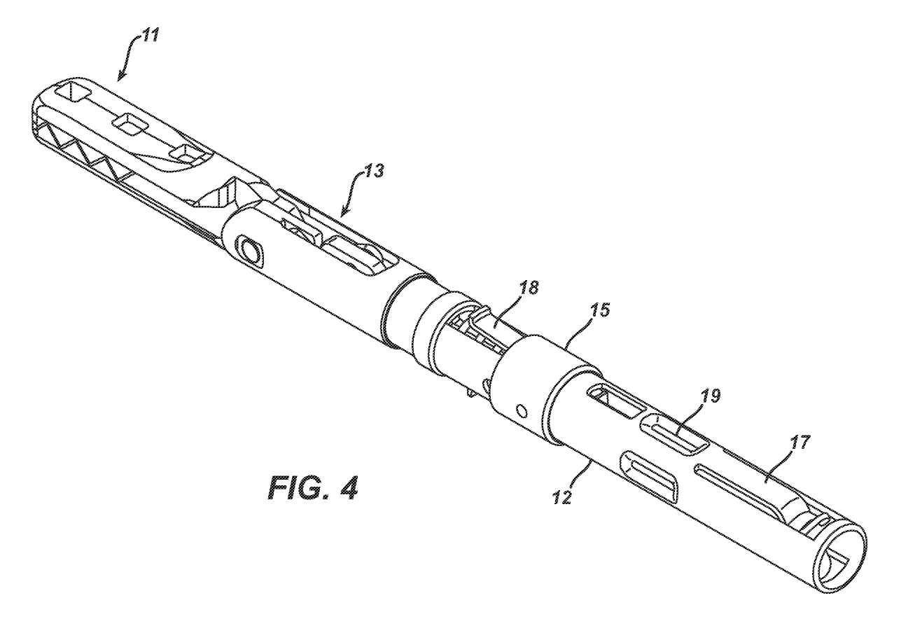

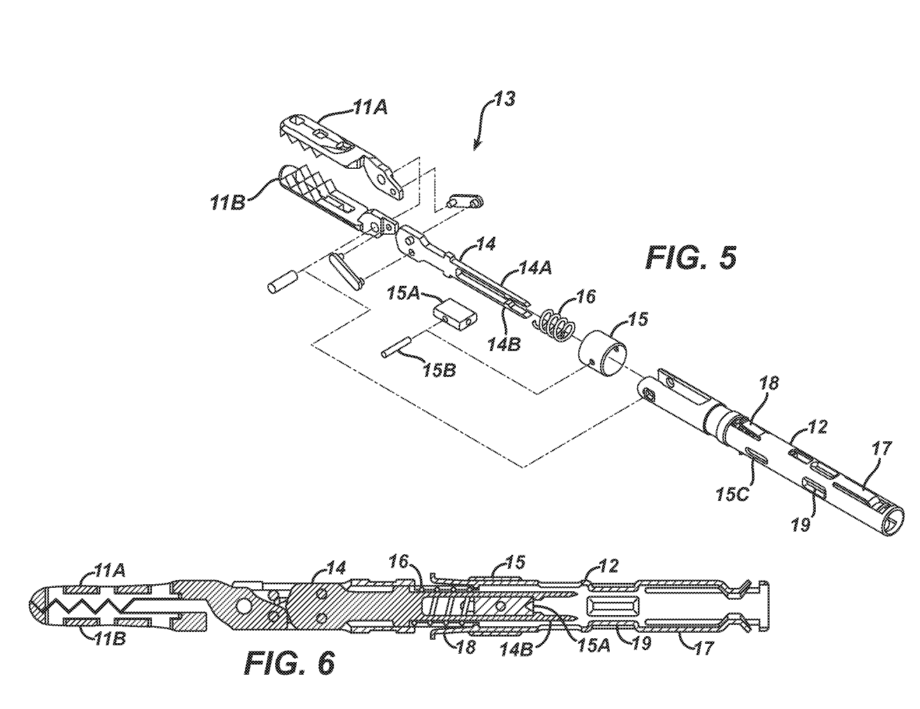

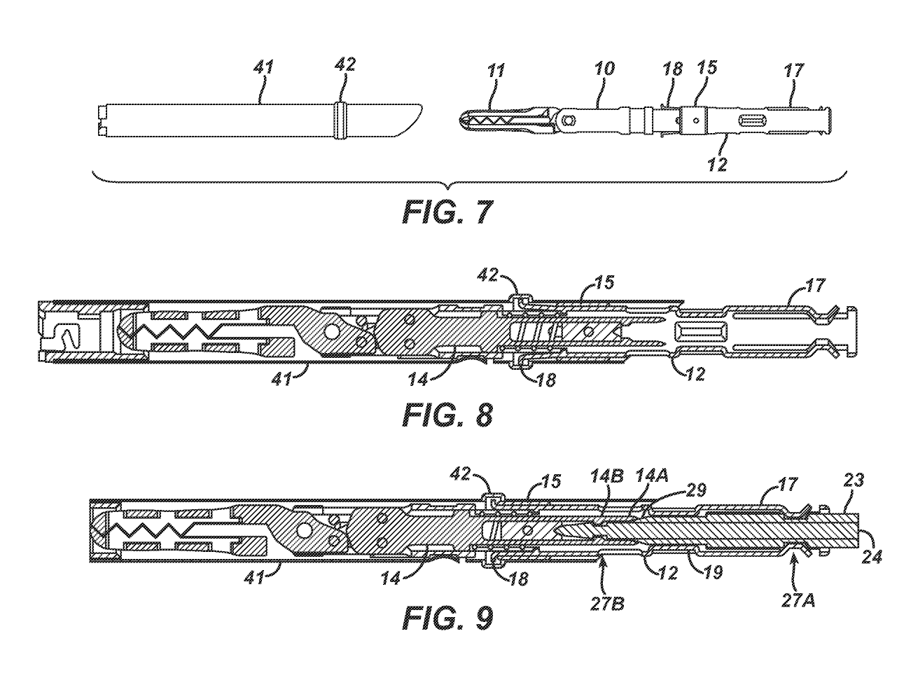

FIGS. 1-7 illustrate one example a laparoscopic surgical instrument system. The elongate shaft (20) comprises an outer tube (23) and an inner rod (24) positioned in the outer tube (23). The elongate shaft comprises a distal end (21) and a proximal end (22). The elongate shaft (20) may be rigid and adapted for insertion into a body cavity through an access device, such a trocar, or through direct percutaneous insertion without an access device. The elongate shaft (20) may also be flexible and sized for insertion through the working channel of a flexible endoscope. The rigid shaft embodiment is well suited for laparoscopic surgical procedures, and the flexible shaft embodiment is well suited for natural orifice intraluminal and natural orifice transluminal procedures.

An actuator (30) is operably connected to the proximal ends (22) of the outer tube (23) and the inner rod (24). The actuator (30) causes the inner rod (24) to move relative the outer tube (23). In this embodiment the actuator (30) is a manual pistol grip handle; however, a variety of other manual actuators could also be used, including a scissor grip handle, a syringe grip handle, endoscopic rotary knobs, and the like. The actuator (30) could also take the form of a robotic interface, such as an DAVINCI puck, a housing comprising gears or pulleys, servomechanisms, and the like.

The end effector (10) is adapted for in vivo attachment to and detachment from the elongate shaft (20). An inner shuttle (14) is axially moveable relative the outer casing (12). The shuttle comprises a mating feature adapted to attach to the distal end of the inner rod (24). The specific mating feature can vary, but in this embodiment the mating feature comprises a detent-type mechanism comprising a pair of proximally oriented prongs (14A), each functioning as leaf spring to bias a medially oriented tooth (14B) to engage the notch (27B) on the inner rod (24).

A pair of surgical jaws (11A, B) have an opened position and closed position dependant on the relative axial position of the shuttle (14) and the outer casing (12). A bar linkage mechanism (13) translates the axial motion of the shuttle (14) to open and close the jaws (11); however, a variety of other known mechanisms can be used to effect operation of the jaws (11). In this embodiment, the jaws (11) are shown as a grasper; however, a variety of other tissue manipulating jaws could also be used, including dissectors, sheers, babcocks, forceps, staplers, clip appliers, and the like. Non-jawed end effectors could also be employed such as hook knives, snares, retractors, and the like. In the case of end effectors that require energy, appropriate energy transmission mechanisms known in the art can be added. For instance, appropriate electrical connections can be added between the shaft (20) and end effector (10) to enable bi-polar forceps. Similarly, an ultrasonic transducer and waveguide can be added for the ultrasonic shears end effector.

The end effector (10) comprises a mating feature (17) adapted to attach the outer casing (12) to the outer tube (23). In this embodiment the mating feature (17) is a detent-type mechanism comprising two leaf springs, positioned 180 degrees from each other on the outer casing (12), each having a medial facing tooth. As the outer tube (23) slides distally into the outer casing (12), and the leaf springs provide a biased snap-to-lock engagement of the teeth into the notch (27A). Other mating features could also be used, including threads, collets, bayonets, and the like. Keyway features (19) are sized and adapted to engage the spline features (28) on the outer tube (23) to facilitate rotational coupling when the outer tube (23) is attached to the outer casing (12). Accordingly, rotational control on the actuator (30) can be used to rotate the end effector (10) during surgical procedures.

The end effector (10) includes a lock (18) moveable between a lateral position and medial position. The lateral position corresponds to the locked position, and the medial position corresponds to the unlocked position. In this embodiment, the lock (18) comprises two laterally oriented prongs, each attached to a leaf spring biasing the respective prong to its lateral position. A ring (15) circumscribes the outer casing and is axially moveable between a proximal position and a distal position. In the distal position, the ring (15) interferes with the leaf springs to constrain and deflect the prong to its medial position. In the proximal position, the ring (15) does not interfere with the leaf springs so the prongs return to their lateral positions. A pusher (15A) is positioned between the shuttle prongs (14A) and is connected to the ring (15) with the pin (15B). The pin (15B) rides in the slot (15C), which limits the axial stroke of the ring/pusher/pin sub-assembly. A compression spring (16) acts on the pusher (15A) and biases the ring (15) to its proximal position. The compression spring (16) also acts on the shuttle (14) biasing it to its distal position and the jaws (11) to their closed position.

A loader (40) comprises a handle (45), a shaft (43), and a distal tube (41) that may articulate relative the shaft (43). The tube (41) is sized to receive the end effector (10). As shown in FIG. 8, an inner circumferential recess (42) is sized and adapted to receive the lock (18) when it is in its lateral position thereby locking the end effector (10) in the tube (41). As shown in FIG. 9, when the lock (18) is in its medial position, the lock (18) does not engage the recess (42) therefore allowing the end effector (10) to leave the tube (41). The tube (41) may also include a leaf spring to engage the end effector (10) and provide some frictional resistance to prevent the end effector (10) from unintentionally exiting the tube (41) when the lock (18) is in its medial position.

During surgery, the distal end (21) of the shaft (20) is inserted into the body cavity, such as the abdomen, pelvis, thorax, etc. The end effector (10) is locked into the loader tube (41) and also introduced into the body cavity, typically through an access port. The loader tube (41) can be articulated to facilitate bringing the end effector (10) into axial alignment with the shaft (20). The distal end (21) is then inserted into the end effector (10). The outer tube (23) slides distally into the outer casing (12) until the leaf springs of the mating feature (17) provide a biased snap-to-lock engagement into the notch (27A), thus attaching the outer casing (12) to the outer tube (23). The inner rod (24) slides distally into the shuttle (14) until the prongs (14A) provide a biased snap-to-lock engagement with the teeth (14B) into the notch (27B), thus attaching the shuttle (14) to the inner rod (24). Insertion of the inner rod (24) into the shuttle (14) simultaneously pushes the plunger (15A) which in turn pushes the ring (15) distally to unlock the lock (18). Thus, the end effector (10) unlocks from the tube (41) only when then inner rod (24) is attached to the end effector (10). The end effector (10) is now fully attached in vivo to the shaft (20) and can then be pulled from the tube (41). The loader (40) can be removed from the surgical field. Operation of the actuator (30) translates to operate the jaws (11), thus enabling a surgeon to manipulate tissue and perform minimally invasive surgical procedures.

In vivo detachment is achieved by reversing the attachment steps outlined in the previous paragraph. The loader (40) is introduced into the body cavity and the end effector (10) is slid into the tube (41). The distal end (21) of the outer tube (23) comprises a sloped surface (29). When the ring (25) is rotated, its distal cam surfaces (25B) act on the pin (25A) thus advancing distally the outer tube (23) and compressing the spring (26). As a result, the sloped surface (29) also advances distally to engage and laterally spread the shuttle prongs (14A) thus disengaging the teeth (14B) from the notch (27B) on the inner rod (24). The inner rod (24) can now be pulled from the shuttle (14) (the sloped surfaces (29) may also be used to facilitate in vivo insertion of the inner rod (24) into the shuttle (14)). Simultaneously, the spring (16) acts on the plunger (15A) to move the ring (15) proximally to lock the lock (18). Thus the end effector (10) detaches from the inner rod (24) only when the end effector (10) is locked in the tube (41). The distal end (21) can then be pulled to overcome the snap-to-lock engagement of the mating feature (17) and liberate the shaft (20) from the end effector (10).

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical device, comprising:
a) an elongate shaft comprising a distal end and a proximal end;
b) an actuator operably connected to the proximal end of the elongate shaft;
c) an end effector adapted for in vivo attachment to and detachment from the distal end of the elongate shaft;
d) a loader sized to receive the end effector; and
e) a lock extending laterally from the end effector, the lock being operative to prevent the end effector from leaving the loader.

2. The surgical device of claim 1, wherein the lock can move medially relative the end effector to allow the end effector to leave the loader.

3. The surgical device of claim 1, wherein the actuator is a manual handle.

4. A surgical device, comprising:
a) an elongate shaft comprising an outer tube and an inner rod positioned in the outer tube, the outer tube and inner rod each comprising a distal end and a proximal end;
b) an actuator operably connected to the proximal ends of the outer tube and the inner rod;
c) an end effector adapted for in vivo attachment to and detachment from the elongate shaft, the end effector comprising:
i) an outer casing comprising a first mating feature adapted to attach to the distal end of the outer tube;
ii) an inner shuttle axially moveable relative the outer casing, the inner shuttle comprising a second mating feature adapted to attach to the distal end of the inner rod;
iii) a pair of surgical jaws having an opened position and closed position dependant on the relative axial position of the shuttle and casing;
iv) a prong moveable between a lateral position and a medial position; and
d) a loader comprising a shaft with a distal tube sized to receive the end effector, the distal tube comprising a recess adapted to receive the prong when it is in its lateral position thereby locking the end effector in the distal tube.

5. The surgical device of claim 4, further comprising ring circumscribing the outer casing, the ring being moveable between a proximal position and a distal position.

6. The surgical device of claim 5, whereby the ring actuates the prong between its lateral position and a medial position.

7. The surgical device of claim 6, further comprising a spring biasing the ring to its proximal position.

8. The surgical device of claim 7, wherein the spring also biases the shuttle distally.

9. The surgical device of claim 5, whereby the ring is actuated by the insertion of the inner rod into the surgical end effector.

10. The surgical device of claim 5, whereby the prong is attached to a leaf spring biasing the prong to its lateral position.

11. The surgical device of claim 4, wherein the first mating feature comprises a medial tooth adapted to engage a notch in the outer tube.

12. The surgical device of claim 4, wherein the second mating feature comprises a medial tooth adapted to engage a notch in the inner rod.

13. The surgical device of claim 4, wherein the second mating feature comprises a pair of proximally oriented prongs each comprising a medially oriented tooth sized and adapted to engage a notch on the inner rod.

14. The surgical device of claim 13, wherein the distal end of the outer tube comprises a sloped surface adapted to laterally spread the proximally oriented prongs of the shuttle, thereby disengaging the medially oriented teeth from the notch on the inner rod.

15. The surgical device of claim 14, further comprising a ring operably connected to the outer tube to advance the sloped surface distally.

16. The surgical device of claim 15, where the ring comprises a distal cam surface acting on a pin that is connected to outer tube, whereby rotating the ring pushed the pin distally thus advancing the sloped surface distally.

17. A surgical device, comprising:
a) an elongate shaft comprising an outer tube and an inner rod positioned in the outer tube, the outer tube and inner rod each comprising a distal end and a proximal end;
b) an actuator operably connected to the proximal ends of the outer tube and the inner rod;
c) a tubular loader sized to receive the end effector, the tubular loader comprising a recess adapted to receive the prong when it is in its lateral position thereby locking the end effector in the tubular loader;
d) an end effector adapted for in vivo attachment to and detachment from the elongate shaft, the end effector comprising:
i) an outer casing and a means to attach the outer casing to the outer tube;
ii) an inner shuttle axially moveable relative the outer casing and a means to attach the shuttle to the inner rod;
iii) a pair of surgical jaws having an opened position and closed position dependant on the relative axial position of the shuttle and casing; and
iv) a means to lock and unlock the end effector in the tubular loader.

18. The surgical device of claim 15, further comprising a means to detach the inner rod from the inner rod from the shuttle.
